(19) United States
(12) Patent Application Publication
(10) Pub. No.: US 2016/0066877 A1
(43) Pub. Date: Mar. 10, 2016

(54) FOREIGN OBJECT DETECTION PROTOCOL SYSTEM AND METHOD

(71) Applicant: RaPID Medical Technologies, LLC, Chicago, IL (US)

(72) Inventors: Vicko Gluncic, Chicago, IL (US); Gady Agam, Naperville, IL (US)

(21) Appl. No.: 14/846,880

(22) Filed: Sep. 7, 2015

Related U.S. Application Data
(63) Continuation of application No. PCT/US15/48742, filed on Sep. 6, 2015.
(60) Provisional application No. 62/046,925, filed on Sep. 6, 2014.

(51) Int. Cl. A61B 6/00; A61B 6/12

(52) U.S. Cl. CPC A61B 6/563; A61B 6/12; A61B 6/488; A61B 2019/5236

(57) ABSTRACT

The present invention relates to the coupling of medical procedures with protocols and advanced software applications, coordinating existing medical procedures and protocols with image detection software to integrate and enhance the identification of IMDs and the detection of RSIs. In one aspect, the emergency room or the operating room protocol is modified so that a scan or XR of the patient is taken for identification of any IMD's. In another aspect, the operating room protocol is modified so that there is an additional step of calibrating an intraoperative or post-operative scans or XR specifically for detecting RSIs and/or identifying IMDs in addition to any scans or XRs performed for the purpose of monitoring the treatment of the patient.

### BACKGROUND OF THE INVENTION

1. Field of the Invention

The invention relates to medical pattern recognition systems and methods. More specifically, but not exclusively, the field of the invention is that of medical protocols for enhancing the ability to detect and remove foreign objects from the body.

2. Description of the Related Art

Techniques are known for the identification of implanted medical devices (“IMD's”) and retained surgical foreign objects (“RSFOs”)/also called retained surgical items (“RSI”) in medical images/or retained foreign objects (RFOs) in a broader meaning.

Approximately 25 million patients in the United States have or have had an implanted medical device (“IMD”). Driven by a rapidly increasing aged population and supported by new technologies, the demand for IMDs and their further proliferation can only be expected to increase.

An IMD is a medical device that is partly or totally surgically inserted into the human body or a natural orifice and is expected to remain implanted for an extended period or may be permanent. IMD's can further be classified either as active, those that use electricity, or passive, and those that do not use electricity. In the US, medical devices are regulated by the FDA and classified into three classes, on basis of risk and the level of regulatory control that is necessary to assure the safety and effectiveness: class I, class II, and class III. Class III devices include devices that generally affect the functioning of vital organs and/or life support systems with very high health risk if the device were to malfunction.

Identification of an IMD during patient admission, and especially in emergencies, is crucial for the safe and efficient management of that patient. Concerns with the accurate and timely identification of IMD's are an emerging safety issue. Of particular concern is the commonly encountered situation where medical records are not available and/or the patient is unable to provide the appropriate information/documentation regarding the IMD he has. Most commonly IMD's are initially reported by patients or noted on admission and/or emergency x-rays (“XR”), magnetic resonance images (“MRI”), ultrasound or computerized tomography (“CT”) images, necessitating, often ineffective, attempts to gather more information regarding the device in question. This usually involves contacting the patient's family, primary care providers or health care institutions previously visited by the patient. Even when such attempts are successful, available information about the patient's device is often incomplete, unreliable and delayed. On the other hand, the large variety, rapidly increasing number approved by Food and Drug Administration (FDA), and difficult projections/orientations of IMD's in medical images (XR, CT, or MRI) make their identification very difficult for physicians/radiology specialists. Possible consequences include: delayed appropriate diagnostic imaging and care, medical complications arising from device incompatibility with imaging or therapeutic modalities, and suboptimal care due to inappropriate avoidance of treatment and diagnostic procedures that are erroneously considered contraindicated.

Software applications facilitate initial assessment/identification, expedite the management, and improve the healthcare and safety of patients with IMD's, including those with symptoms of IMD malfunction. They also facilitate implementation of recent FDA requirements for post-market device surveillance.

Physicians are increasingly encountering patients with IMD's. Identification of an IMD, during an emergent admissions in particular, is critical for safe and efficient patient management. In 2007, FDA issued a report indicating an increase in adverse events linked to medical devices, including 2,830 deaths, 116,086 injuries, and 96,485 device malfunctions. Class III active IMD's were cited in a relatively high number of fatality reports within the FDA report.

Ultra-low-power radio-frequency (RF) technology has greatly facilitated the development of IMD's. The ability to wirelessly transmit the patient's and IMD's data enables a clinician to obtain useful diagnostic information and reprogram therapeutic settings. Furthermore, radio-frequency identification (RFID) technology uses radio waves to transfer data from an electronic tag to identify and track the tagged device. However, the rapidly increasing number of IMD's and their manufacturers, absence of the standardized tools/methods capable of RF sensing, identifying, and reprogramming IMD's, radio interference problems, ethical/security issues, and the fact that many IMD's do not have RF capabilities make this technology less convenient for rapid identification. This disadvantage is particularly obvious in medical emergencies and emergency room settings.

Medical errors involving IMDs, especially those arising from their incompatibility with treatment or diagnostic procedures, are an emerging patient safety issue. Procedures incompatible with patient's devices have been performed, leading to device malfunction and other complications. Examples of such complications include: patients undergoing Magnetic Resonance Imaging (MRI) in the presence of implanted ferromagnetic devices possibly causing migration, interference with the function of implanted devices because of strong magnetic fields (MR) and disrupting electrical forces (certain types of CT or surgical electro cautery). This includes setting changes of active (none turned off) cardiac pacemakers and defibrillators and/or defibrillation shocks during surgical procedures caused by electro cautery scalpels. In another example, percutaneous catheters and ports have been damaged by exceeding their pressure ratings during therapeutic infusions, necessitating subsequent surgical interventions/exchange or repair. Furthermore, several IMD's are compatible with MRI and CT imaging but/and/or requires reprogramming after the completion of the MRI that has been frequently missed. These effects on the IMD are not always evident or immediately observed (such as unintended re-programming, e.g., ventricular-peritoneal shunts' valves) and can not only lead to delays but also to serious and possibly disastrous complications. Conversely, there are patients that do not receive optimal treatment and diagnostic procedures, even though their devices are compatible with such treatments. For example, several pacemakers currently on the market are compatible with MRI. In these cases, disclosure software identifies these specific models as being compatible with MRI, providing the treating physicians an option to have their patient undergo a medically-indicated MRI scan safely.

Retained surgical item (RSI) is any object unintentionally left in patients during the surgery. RSIs include needles and surgical instruments and/or materials, and continue to be a significant problem with an incidence of between 0.3 and 1.0 per 1,000 surgeries. This has resulted in a significant increase in patient care costs and consecutive legal expenses.

Intra-operative or early post-operative identification of RSIs is critical for safe and efficient management of surgical patients. Current recommendations for prevention of RSIs in the operating room (“OR”) include methodical wound exploration before closing, usage of standardized practices for surgical items accounting, usage of items with radiopaque markers within the operative site, and mandatory operative field XRs before wound closure when a item count discrepancy occurs. In addition, radiographic screening is recommended at the end of an emergent surgical procedure, unexpected change in the procedure, and for patients with a high body mass index. Some institutions also conduct routine postoperative screening XRs for the prevention of RFOs. Therefore portable XR radiological protocols have become crucial for timely RSIs detection. However, they have relatively low efficacy and require significant time for completion and for evaluation. The underlying problems of their use are the relatively low sensitivity and specificity of the human eye in the identification of relatively small objects in a large XR field and the fact that radiologists and surgeons do not routinely undertake formal training in the recognition of RSIs.

Technological aids to assist the OR team in the detection and prevention of retained sponges, gauze towels, and laparotomy pads include radio-frequency detectable sponge systems and bar-coded sponge systems. These aids are intended to augment the standardized manual count practices, and to not replace them.

Operative field XR is mandatory when there is a counting discrepancy of surgical instruments or materials at the end of the procedure. According to the 2006 Patient Care Memorandum of the Department of Veterans Affairs (Boston Healthcare System, VA, USA), surgical instruments and/or materials must be counted, except for procedures that are routinely concluded with a radiograph (for example, an orthopedic case to assure proper alignment of a bone or implant). In these cases, XR is mandatory if an instrument count is not performed, and the evaluation of the XR must be performed before the patient is transferred from the OR to determine whether any instruments or sponge has been retained. When a radiograph is requested to locate a missing item, the type of foreign object that is missing, OR number, and telephone number must be specified in the request to the radiologist. XR screening is also recommended/mandatory at the end of emergent surgical procedures, unexpected changes in procedures, or in patients with high BMI. Some institutions use postoperative screening XRs routinely. In all of these cases, the completion of the surgical case may be delayed until radiologic evaluation is received. Assuming the patient is stable, current recommendations are that in the event of an incorrect count, a XR of the operative field should be made available to a radiologist within 20 minutes and their evaluation/confirmation of the results of the XR should be provided back to the OR within another 20 minutes. This process frequently takes significantly more time than 40 minutes.

Portable XR is also a method of choice for determination of the relative position/location of a RSI. This is particularly important if the specific tissue layer or surgical incision/wound is already closed and additional instruments are present in the XR image.

While stainless steel instruments are likely to be detected successfully on XR screening, XRs are less sensitive in detecting sponges and needles. Sponges may be difficult to detect because they may become twisted or folded, distorting visualization of the radiopaque marker. Needles may also be difficult to visualize due to their size. The value of intraoperative and/or post-operative XR images for RSIs identification has been controversial and very few studies have been undertaken to evaluate their effectiveness. A recent study evaluating portable XR for identification of retained suture needles in ophthalmologic surgical cases showed that the overall sensitivity and specificity of the physicians' review of radiographs with suspected retained needles was 54% and 77%, respectively. This is particularly worrisome considering that in this particular case the size of the surgical field was small, the area of interest well-defined, while the participants in the study have known that they were looking for the needles which should have greatly facilitated RSI/needle detection. In the most studies when XRs were falsely negative for RSI detection; poor-quality radiographs, multiple foreign objects in the field, and failure to communicate the purpose of the radiograph to the interpreting radiologist were cited as contributing factors. Although it is mandatory that such intra-operative radiographs be reviewed by a radiologist(s) and/or surgeon(s), it is not routine for those individual to have undertaken specific/formal training in the radiographic identification/recognition of these objects. Furthermore, the general consensus throughout the literature is that the most effective means of evaluating the presence of a RSI is through the use of CT scanning which—in most of the cases—is not possible in the OR.

The known pertinent and pre-existing technologies include picture archiving and communication system (PACS) which is classified under medical devices by the US FDA. Several companies produce PACS software including Siemens, Philips, GE etc. Moreover, PACSs include software servers and Digital Imaging and Communication in Medicine (DICOM) image archival database that stores patient health care information and x-ray images for use by health care personnel. However, PACSs systems today don't have ability to detect RSIs or identify IMDs in images of any imaging modality.

To prevent RSis, patient safety measures include an effective operative room communication; mandatory counts of surgical instruments and sponges, methodical wound examinations, and XR imaging. Mistakes in counts happen in up to 12.5% of surgeries frequently prompting mandatory XR of the surgical field to rule out RSI. Due to these concerns many hospital systems nowadays mandate XR at the end of the complex surgeries.

The problems in the current process of preventing RSIs are the deceptively correct count—72% to 88% of retained items happen in surgeries with “correct counts”, relatively low sensitivity (approx. 50-70%) of human eye for RSIs in XR images when they are taken, and relatively long time to complete radiological analysis/reading (approx. 20-40 min). Surgical count boards and radio tagged sponges have decreased the incidence of retained sponges however needles (especially those <15 mm long) remain a significant problem. Therefore, current XR protocols for prevention of RSIs are time consuming and not as effective as they should/could be.

In addition, more than 25 million patients in US have critical life functions supported by IMDs. Today there are more than 5000 IMDs on the market such as pacemakers, defibrillators, vagal nerve stimulators etc. Upon patient admission, IMDs are frequently initially reported by patients or noted on medical radiological images, necessitating often-ineffective attempts to determine the specific type of IMD. Each year more than 2,000 deaths occur due to mismanagement of IMDs such as pacemakers, insulin pumps, and others. Currently, there is no universal solution for the identification of IMDs—mandated by Congress in 2007 but still not yet in place.

### SUMMARY OF THE INVENTION

Embodiments of the present invention involve novel XR protocols that have unique combinations of specific steps/methods to optimize detection of RSIs and identification of IMDs by in clinical settings—process which has been termed “RaPID Response X-ray”.

The invention, in several embodiments, provides a quality assurance and patient safety platform including healthcare software for the detection of RSIs or identification of IMDs in radiological images integrated with hospitals' PACSs or stand-alone application available through the hospital electronic medical record interface. The platform aids medical experts analyzing radiological images when searching for RSIs or when trying to identify IMDs. Specific algorithms may be used to enhance and improve the detection of RSIs and/or identification of IMDs, but the specific step of obtaining a XR image particularly for the detection of RSIs has yet to be implemented prior to the invention. Another aspect of platform's embodiments is the identification of IMDs.

In addition to Computer Aided Detection (CADe) software—in order to make the process of RSIs detection or IMDs identification ultimately effective—the embodiments of the invention provide a specific workflow process—developed by using the business process modeling methods—which has been termed “RaPID Response X-ray.”

Other aspects of embodiments of the invention involve usage of CADe software in combination with: X-ray plate with telecommunication/Wi-Fi capabilities usage for the purpose of shortening the time for image transfer to PACS, and specific settings (kV and mAs) of the portable XR machine optimized for RSIs detection or IMDs identification.

In one embodiment of the invention, the insertion of specific textual denominators into the image (for example RSI/needle label if needle counts are not matching or IMD label if IMD is suspected and further specification is needed) is based on what information is needed on the portable XR machine before the image is being taken and uploaded to PACS. In another embodiment, optimization of the PACSs flow to automatically put on the top of the radiologist's work list images with these specific denominators is provided. Still another embodiment provides automatic critical information feedback/alert if CADe detects RSIs or automatic IMD information insertion into electronic medical record if IMD is identified. This process significantly shortens the time necessary for radiological detection of RSIs or identification of IMD's and improves accuracy of the process.

Once the RaPID Response X-ray is ordered the XR technician is provided with portable XR machine and XR plate with telecommunications/Wi-Fi capabilities in order for images to get instantaneously uploaded to PACS. This eliminates timely process of feeding a plate into the reader manually which is frequently not immediately available next to the OR suite or emergency department. Once the portable XR machine is in the operating room and wireless XR plate is in the appropriate position beneath the patient the specific settings of the portable XR machine (including kV and mAs) should be applied rather then using standard setting for chest or abdomen XRs. These RSIs specific settings increase the image quality/contrast for detection of an IMD or RFO. Therefore, any deviation from the standard XR settings such as chest (CXR) or abdomen XR (KUB) predetermined settings with the intention to provide better contrast for identification is part of this process. The ranges of XR settings are based on patient and physical characteristics of the RSI and IMD and demonstrated data. In addition, before the image is taken specific textual image denominator will be assigned to the image—alerting physicians who will analyze image about RSI or IMD and providing information regarding the type of miscount/needle, sponge, or instrument, OR phone call back number, OR front desk pager, and surgeon's pager. This eliminates need for the phone call from the OR or ED to the radiologist specifying what we are searching for. Once the image is been taken—specific optimization of the PACSs workflow automatically puts on the top of the radiologist's list images file with RFO denominator and visible RFO alert.

Usage of CADe software solutions for RSIs detection improves accuracy of detection and decreases time needed for image analysis by physician/radiologist. Computer vision is in many respects superior to the human eye in the detection of defined objects. Embodiments of the invention employ CADe software that is based on complex pattern recognition algorithms—combining elements of artificial intelligence with digital image processing—to detect RSIs on medical images. This system analyzes all the images and if any RSI is detected or IMD is identified inserts alert sign over the area of the image with suspected RFO/IMD.

If a radiologist's analysis is congruent with the CADe software findings—positively detecting RSI or identifying IMD an effective critical information alert is conveyed—radiologist will call the number assigned to the image while the software also triggers a pager alert sent to OR control desk and attending physician/surgeon. In addition embodiments of the software automatically provide links in patient's EMR to the specific PACS images so that OR circulating nurse may pull specific set of images on the computer screen in the OR quickly. In this way a surgeon may get better orientation clues where in the operating field the RSI is located.

In one embodiment, the inventive software platform is not integrated into the PACS system but available on a portable X-ray machine or as a separate application—allowing the physician to have the option to activate for analysis. Once the technician arrives to take X-ray plate that stores the DICOM image, the technician asks: “Would you like a RAPID Response X-ray?” giving the option for the physician to choose. When the image is taken through RaPID Response X-ray process, analyzed through RaPID CADe application, and returned to portable XR machine screen, the operating room or emergency department physician or radiologist, the image is stamped with the RaPID Response X-ray logo along with any detections/identifications.

In another embodiment of the invention, the RaPID patient safety and quality assurance platform/CADe software is integrated with the PACS. Images taken under the RaPID Response X-ray process are automatically stamped with RaPID's logo and any identifications/detections are embedded in the image. The above-described process is thus routinely used when searching for the RSIs or trying to identify IMDs.

In yet another embodiment, in the Emergency Room (ER) setting, any of the two proceeding embodiments may be used. For example: a patient arrives and the physicians are in need of identifying the patient's IMD. If the RaPID patient safety and quality assurance platform/CADe software is not integrated within the PACS the first embodiment may apply and if integrated with the PACS the second embodiment may apply.

The present invention, in another form, may be used for intraoperative identification of previously implanted IMDs such as in the cases of complex orthopedic procedures replacing the existing hardware in the patient.

The present invention, in another form, may be used for the future assessment of complex robotic machinery/robots/humanoid robots/bionic robots/bionic human parts if they have specific modules replaced or upgraded. XR or some other imaging modality may still be the fastest method to determine these parts by using methods and systems proposed in our current and previous application.

The present invention, in another form, may be used to determine whether IMD is counterfeited or original. XR or some other imaging modality may still be the fastest method to determine this by using methods and systems proposed in our current and previous application. This is becoming emerging patient safety issues as many patients are receiving counterfeited low or unacceptable quality IMDs aboard. In addition, assessment whether IMD is real or forged may be crucial for transportation safety. Existing XR scanners at the airports etc. may be upgraded with our software solutions and use slightly modified imaging process to determine counterfeited IMDs in passengers that possibly may be a security treat such as an implanted explosive device.

The present invention is also a method for more safe patient management and more effective OR time utilization as proposed combination of steps leads to faster interpretation of the intraoperative XR images even if some of the steps in the process are not available such as CADe software.

Current RSI X-ray detection protocol: In the current protocol for the RSI management—once the miscounts happens—the OR nurses communicate this finding to the OR team and request the surgeon to explore the operation field and search for the missing item—while they perform another recount. If the recount confirms the missing item and surgeons don't find it they call the X-ray technician to take the X-ray of the operation field. X-ray technician comes and takes the X-ray of the operational field by using standard settings for the chest (if thy operate in thorax) or abdomen (if they operate in abdomen) X-rays. Once the X-ray is taken, the technician takes the plate to the plate-reading machine, which uploads image to the PACS. If the X-ray machine has capabilities to show image on the screen—surgeons also try to identify the RSI in the image while waiting for the radiologists report. In the same time the OR circulating nurse should call the radiologist on call and convey the urgency of this particular X-ray, specify to the radiologist what item is apparently missing, and provide a call back number for him to call once he complete image analysis. Radiologist then identifies the image on the workflow list, analyze it, and report back to the OR. The same process is pertinent in the case of the emergent or complex surgeries when the operational field X-ray is mandatory—except this time the OR circulating nurse communicates to the radiologist that clearance for RSIs is needed rather then specifying the missing object. This process takes approximately 20-40 minutes to complete.

RaPID Response X-ray RSI detection protocol: Once the miscounts happens—the OR nurses communicate this finding to the OR team and request the surgeon to explore the surgical wound and the operation field and search for the missing item—while they perform another recount. If the recount confirms the missing item and surgeons don't find it they call the X-ray technician to take the RaPID Response X-ray of the operational field. Once in the OR, the technician has X-ray plate with Wi-Fi capabilities (which will automatically upload images to PACS as soon as they are taken rather then caring the plates to the reader machine) in the appropriate position, the specific settings of the X-ray machine (including kV and mAs) are applied (rather then using standard setting for chest or abdomen X-ray). These settings increase the chance of high quality contrast image of the RSI. In addition, the specific textual image denominator will be assigned to the image —alerting physicians who will analyze image about emergent RSI suspicion—providing information regarding the type of miscount/needle, sponge, or instrument/and OR phone call back number with surgeon's pager. Once the image has been taken, specific optimization of the PACSs flow will automatically put it on the top of the radiologist's list images file with RSI specific denominator and alert. This will eliminate need for the phone call from the OR to the radiologist alerting that we need emergent analysis and specifying what is searching for and automatically put the X-rays on the top of the radiologist's workflow. Once the images are uploaded to the PACS, automatic computer assisted detection (CADe) software solutions for RSIs detection assists and improves radiologist's accuracy of detection and decreases time needed for image analysis. This system analyzes images and, if RSI is detected, inserts alert sign over the specific area of the image with suspected RSI. If software detects the RSI—it also automatically shows the X-ray image with alert sign over the area of the image with suspected RSI on computer screen in the OR while waiting for radiologist detection. If radiologist's analysis is congruent with the CADe's software findings—positively detecting RSI an effective critical information alert will be conveyed—the radiologist calls the number assigned to the image while software—upon radiologist confirmation of the findings by mouse click on automatic conformation function embedded in image together with alert sign—also triggers a pager alert to be sent to OR control desk and attending surgeon. If radiologist's analysis is congruent with the CADe's software negative findings—the clearance information is conveyed in the same or similar manner. The same, or similar, process is pertinent in the case of the emergent or complex surgeries when the operational field X-ray is mandatory—except this time the X-ray denominator communicates to the radiologist that clearance for RSIs is needed rather then specifying the missing object. This way organized process my provide feedback to the OR within approximately 2-5 minutes.

Differences between current RSI X-ray detection protocol and RaPID Response X-ray RSI detection protocol involve the usage of one or more, alone or in combination, of the following:
X-ray plate with Wi-Fi capabilities
Specific (kV and mAs) settings of the portable X-ray machine according to the RaPID's protocol
Preformed specific textual X-ray image denominators available at portable X-ray machine that will be embed in in the comment section. For example: If miscount was for reytec sponge the comment section will have
RSI: reytec sponge
OR call back number: XXX
Circulating nurse pager number: XXXX
OR front desk number: XXXX
Specific optimization of the PACSs flow that automatically puts the image on the top of the radiologist's workflow list as an emergency
Computer assisted detection (CADe) software

Effective organized critical information alert on several levels upon identification These process changes not only decrease time to rule out RSI but also increase accuracy of the information flow and RSIs' detection.

Further aspects of the present invention involve usage of any specific XR machine settings and CADe software to asses/identify IMDs for counterfeiting purposes—such as determining whether IMD in the passenger boarding the airplane is real or fake on security XR screening.

### BRIEF DESCRIPTION OF THE DRAWINGS

The above mentioned and other features and objects of this invention, either alone or in combinations of two or more, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized in conjunction with embodiments of the present invention.

FIG. 3 is a flow chart diagram of the operation of an embodiment of the present invention.

FIG. 4 is a schematic diagrammatic view of operational hospital imaging systems involved with embodiments of the invention.

Corresponding reference characters indicate corresponding parts throughout the several views. Although the drawings represent embodiments of the present invention, the drawings are not necessarily to scale and certain features may be exaggerated in order to better illustrate and explain the full scope of the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiment disclosed below is not intended to be exhaustive or limit the invention to the precise form disclosed in the following detailed description. Rather, the embodiment is chosen and described so that others skilled in the art may utilize its teachings.

The detailed descriptions that follow are presented in part in terms of algorithms and symbolic representations of operations on data bits within a computer memory representing alphanumeric characters or other information. A computer generally includes a processor for executing instructions and memory for storing instructions and data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of embodiments of the present invention; the operations are machine operations. Useful machines for performing the operations of one or more embodiments of the present invention include general purpose digital computers or other similar devices. In all cases the distinction between the method operations in operating a computer and the method of computation itself should be recognized. One or more embodiments of the various embodiments of present invention relate to methods and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

One or more embodiments of the present invention also relate to an apparatus for performing these operations. This apparatus may be specifically constructed for the required purposes or it may comprise a general purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general purpose machines may be used with programs written in accordance with the teachings herein, or it may prove more convenient to construct more specialized apparatus to perform the required method steps. The required structure for a variety of these machines will appear from the description below.

One or more embodiments of the present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system can be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “object” relates to a set of computer instructions and associated data which can be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers which are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks can access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program that carries out one or more specific functions and may be used alone or combined with other modules of the same system or program.

The term “desktop” means a specific user interface which presents a menu or display of objects with associated settings for the user associated with the desktop. When the desktop accesses a network resource, which typically requires an application program to execute on the remote server, the desktop calls an Application Program Interface (“API”), to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a world wide network of computers, namely the “World Wide Web” or simply the “Web”. Examples of Browsers compatible with on or more embodiments of the present invention include the Chrome browser program developed by Google Inc. of Mountain View, Calif. (Chrome is a trademark of Google Inc.), the Safari browser program developed by Apple Inc. of Cupertino, Calif. (Safari is a registered trademark of Apple Inc.), Internet Explorer program developed by Microsoft Corporation (Internet Explorer is a trademark of Microsoft Corporation), the Opera browser program created by Opera Software ASA, or the Firefox browser program distributed by the Mozilla Foundation (Firefox is a registered trademark of the Mozilla Foundation). Although the following description details such operations in terms of a graphic user interface of a Browser, one or more embodiments of the present invention may be practiced with text based interfaces, or even with voice or visually activated interfaces, that have many of the functions of a graphic based Browser.

Browsers display information which is formatted in a Standard Generalized Markup Language (“SGML”) or a HyperText Markup Language (“HTML”), both being scripting languages which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).[0060] Similarly, JavaScript Object Notation (JSON) may be used to convert between data file formats.

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD″) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

The terms “x-ray”, “image” or “scan” or derivatives thereof refer to x-ray (XR), magnetic resonance imaging (MRI), computerized tomography (CT), sonography, cone beam computerized tomography (CBCT), or any system that produces a quantitative spatial representation of a patient or object. “PACS” refers to Picture Archiving and Communication System (PACS) involving medical imaging technology for storage of, and convenient access to, images from multiple source machine types. Electronic images and reports are transmitted digitally via PACS; this eliminates the need to manually file, retrieve, or transport film jackets. The universal format for PACS image storage and transfer is DICOM (Digital Imaging and Communications in Medicine). Non-image data, such as scanned documents, may be incorporated using consumer industry standard formats like PDF (Portable Document Format), once encapsulated in DICOM. A PACS typically consists of four major components: imaging modalities such as X-ray computed tomography (CT) and magnetic resonance imaging (MRI) (although other modalities such as ultrasound (US), positron emission tomography (PET), endoscopy (ES), mammograms (MG), Digital radiography (DR), computed radiography (CR), etc. may be included), a secured network for the transmission of patient information, workstations and mobile devices for interpreting and reviewing images, and archives for the storage and retrieval of images and reports. When used in a more generic sense, PACS may refer to any image storage and retrieval system.

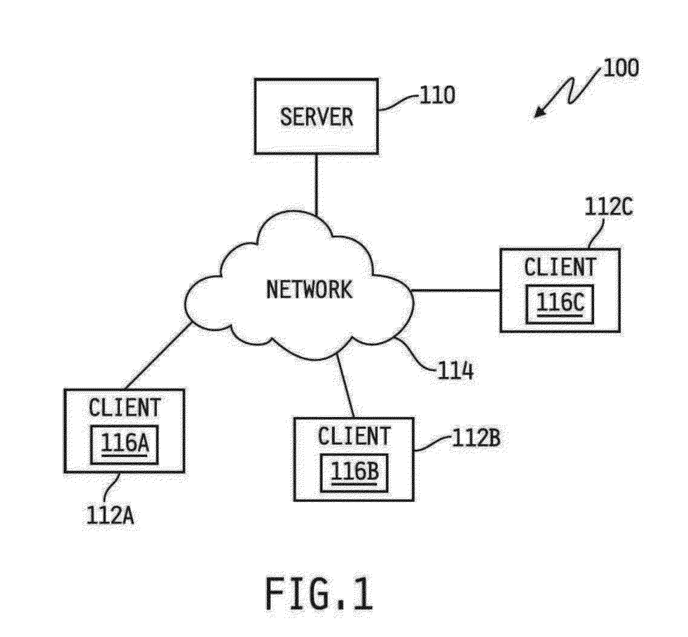

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

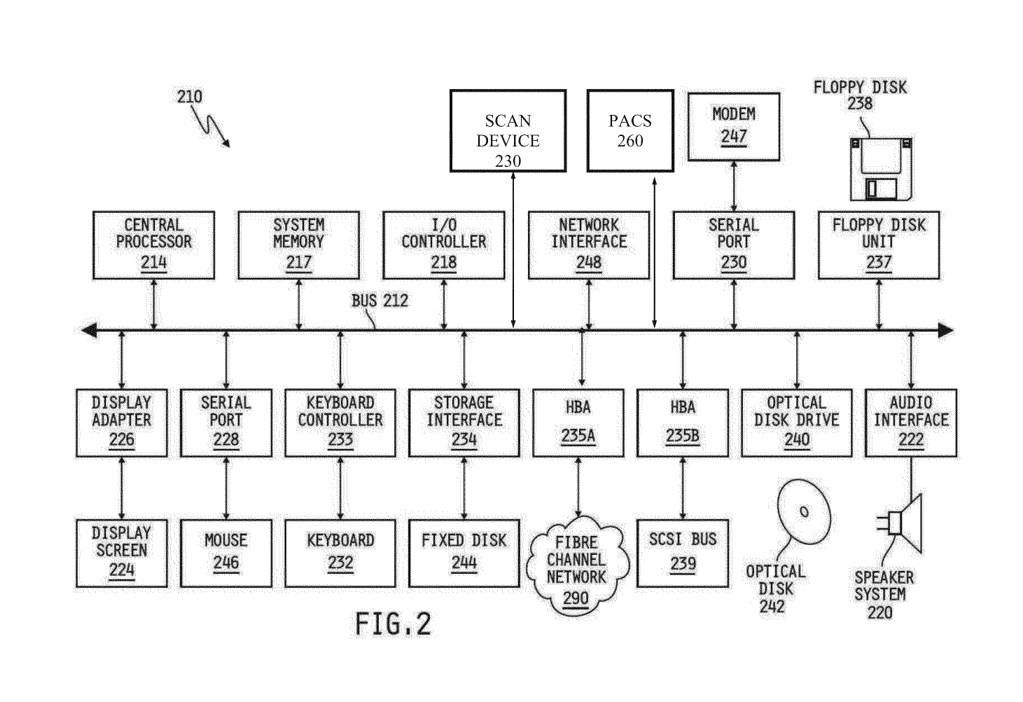

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS) which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like. For example, while scan device 230 (e.g., an x-ray machine, ultrasound, etc.) and/or PACS 260 may be directly connected to bus 212, alternatively such systems may be accessed through network interface 248.

Many other devices or subsystems (not shown) may be connected in a similar manner (e.g., document scanners, digital cameras and so on). Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. In health care environments, the system of FIG. 2 may optionally include scan device 230 (such as an x-ray machine, ultrasonic scanner, or MRI) and may have a connection with PACS 260. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), or other known or developed operating system. In some embodiments, computer system 210 may take the form of a tablet computer, typically in the form of a large display screen operated by touching the screen. In tablet computer alternative embodiments, the operating system may be iOS® (iOS is a registered trademark of Cisco Systems, Inc. of San Jose, Calif., used under license by Apple Corporation of Cupertino, Calif.), Android® (Android is a trademark of Google Inc. of Mountain View, Calif.), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

One or more embodiments of the present invention relate to the coupling of medical procedures with protocols and advanced software applications. The previously described problems with RSIs and/or IMDs have existed for decades, as have pattern detection algorithms and software. Embodiments of the invention specifically coordinate existing medical procedures and protocols with image detection software to integrate and enhance the identification of IMD's and the detection of RSIs that had previously been unattainable. In one aspect, the emergency room or the operating room protocol is modified so that a scan or XR is taken of the patient for identification of any IMD. In another aspect, the operating room protocol is modified so that there is an additional step of calibrating an intraoperative or post-operative scans or XR specifically for detecting RSIs in addition to any scans or XR performed for the purpose of monitoring the treatment of the patient. Departing from conventional post-operative scans or XRs, according to embodiments of the present invention the scan or XR is made under conditions that optimize the detection of RSIs or identification of IMDs. Conventional image detecting algorithms, using computing machinery described above or equivalent processors may be used with embodiments of the invention, and such conventional image detecting algorithms and/or improved image detecting algorithms developed in conjunction with embodiments of the invention to enhance the identification of IMDs and detection of RSIs using conventional XR and computing equipment.

To prevent RSIs, patient safety measures include an effective operative room communication; mandatory counts of surgical instruments and sponges, methodical wound examinations, and XR imaging. Mistakes in counts conventionally happen in up to 12.5% of surgeries prompting mandatory x-ray of the surgical field to rule out any RSIs. Due to these concerns many hospital systems nowadays mandate XR at the end of the complex surgeries including all emergent surgical procedures.

Problems in the conventional process of preventing RSIs are the deceptively correct count—72% to 88% of retained items happen in surgeries with “correct counts”, relatively low sensitivity (approx. 50-70%) of human eye for RSIs in XR images when they are taken, and relatively long time to complete radiological analysis/reading (approx. 20-40 min). Surgical count boards and radio tagged sponges have decreased the incidence of retained sponges however needles (especially those <15 mm long) remain a significant problem. Therefore, conventional XR protocols for prevention of RSIs are time consuming and not as effective as they should/could be.

In addition, more than 25 million patients in US have critical life functions supported by IMDs. Today there are more than 5000 IMDs on the market such as pacemakers, defibrillators, vagal nerve stimulators etc. Upon patient admission, IMD's are frequently initially reported by patients or noted on medical radiological images, necessitating often-ineffective attempts to determine the specific type of IMD. Each year more than 2,000 deaths occur due to mismanagement of IMDs such as pacemakers, insulin pumps, and others. Currently, there is no universal solution for the identification of IMDs. RaPID quality assurance and patient safety platform provides an effective paradigm for a unique IMDs identification system—mandated by Congress in 2007 but still is not in place.

Further, conventional PACSs are used to manage radiological workflow. However, conventional workflow does not use alert systems to identify patients waiting for XR results in the operating room during surgical procedures specifically waiting for radiology personnel to determine if a RSI is left within the body or to identify the IMD resulting in a delays within the surgical department/ORs. The conventional protocol requires individual radiological technicians to call the radiologist who may be home or off-duty for examination of a DICOM image. This process may take up to an hour and decreases the efficiency of the OR turnover protocols—the efficiency of delivering care from one patient to the next.

RSIs are typically any surgical tool or sponge inadvertently left behind in a patient's body in the course of surgery. Approximately two-thirds of the RSIs are surgical sponges, and other third represents mostly surgical needles and less frequently surgical instruments. The consequences of RSIs include injury, repeated surgery, prolonged hospital stay, excess monetary cost, loss of hospital credibility, and death of the patient.

The U.S. Center for Disease Control and Prevention has estimated the total number of surgeries performed in 2010 has exceeded 100,000,000. According to the estimate of the U.S. Department of Health and Human Services, the frequency of RSIs are currently between 0.02-1% of all surgeries.

Problem in the conventional process of preventing RSIs include the deceptively correct count, where 72% to 88% of retained items happen in surgeries with “correct counts”, relatively low sensitivity (approx. 50-70%) of human eye for RSIs in XR images when they are taken, lack of formal training for radiologists and surgeons to identify RFO's in the x-ray images, and the relatively long time to complete radiological analysis/reading (approx. 20-40 min).

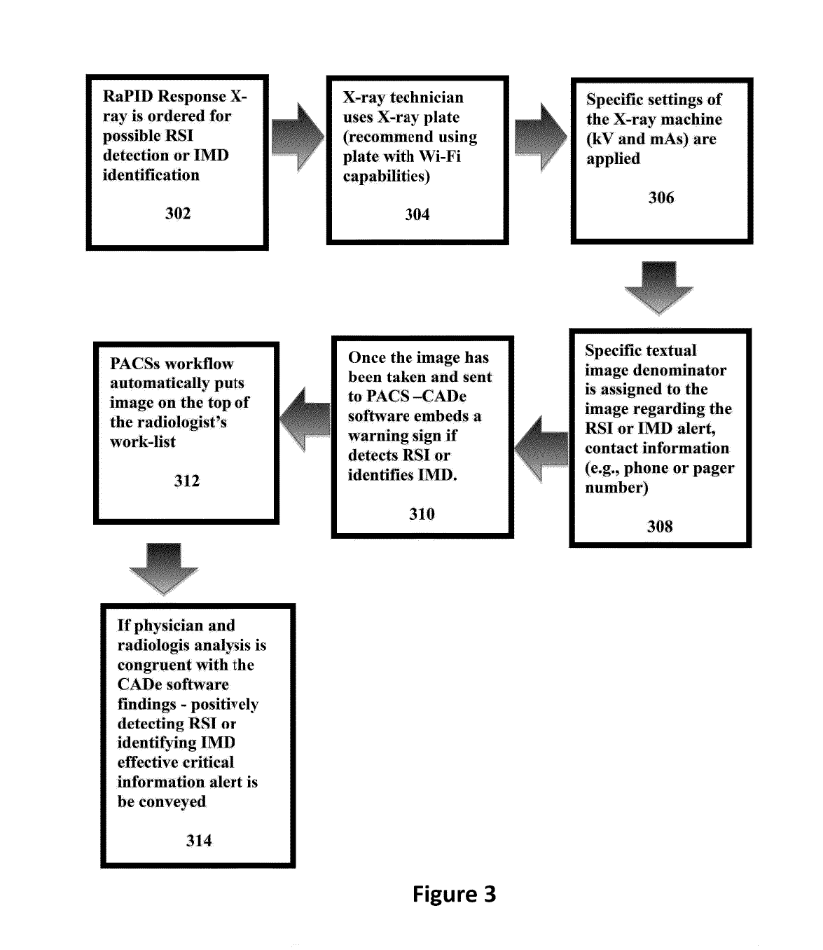

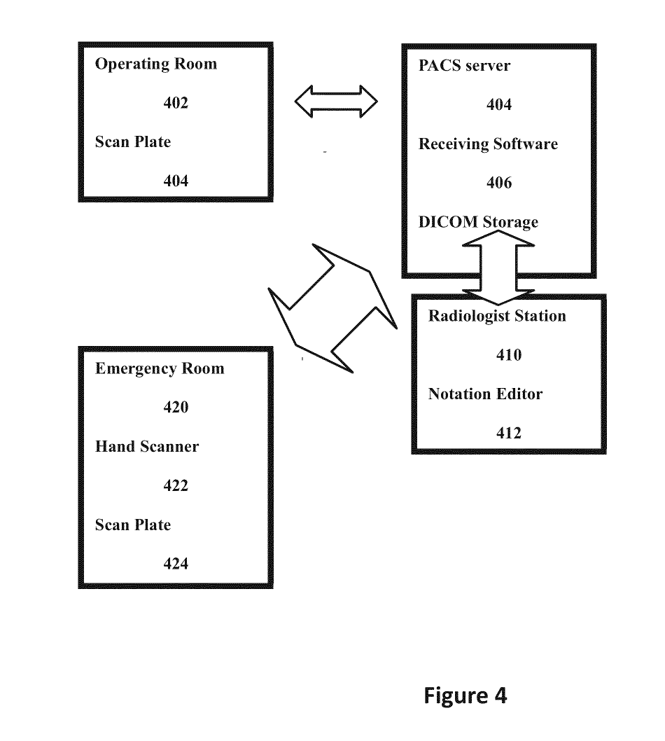

The general protocol of one embodiment of the present invention is shown in FIG. 3, which may be altered or modified for a particular situation or implementation, and integrated into a larger hospital and/or surgical protocols in hospitals, clinics, emergency centers and the like. In step 302 the RaPID response X-ray is ordered. Many specifics of this step are mentioned in the descriptions of various embodiments in this document. In step 304, the technician uses the X-ray plate, and it is recommended to have the X-ray plate outfitted with WiFi capabilities to enhance image processing speed. However, it is possible in other embodiments that the X-ray plate is otherwise electronically coupled with other image processing systems including PACS server 404 (see discussion of FIG. 4). While not recommended, it is also possible to use an X-ray plate that is not electronic, however such an image would need to be quickly digitized and communicated to PACS server 404.

In step 306, specific settings of the X-ray machine are applied for the purpose of detecting foreign objects in the body rather than for diagnostic purposes. Because RSIs are fainter on most scans than most anatomical objects, the inventors have discovered that detection is enhanced when the settings of the X-ray machine are made with a lower power to enhance the contrast in regions where RSIs are likely to be disposed. In addition, many parts of IMDs, particularly those parts that help distinguish between similarly configured IMDs, are likewise difficult to ascertain from diagnostic scans, so that the lower power settings enhance IMD detection. In step 308, optionally any RSI and/or IMD information and/or contact information is coupled to the image taken, which may be embedded in the image data structure or attached or otherwise associated with the image.

In step 310, in the process of sending the image to PACS server 404, computer aided decision support software (CADe) also processes the image data searching for potential RSIs and IMDs. This CADe software may be present in the originating locations (e.g., operating room 402 or emergency room 420), and optionally the results of the CADe software may be presented in the originating location. Although recommended that any results of the CADe software be evaluated by a physician and radiologist, in some embodiments of the invention, the results may be used immediately, for example in remote locations without radiological support or in emergency situations where external communications are difficult or impossible. A warning sign may be provided, such as a textual alert specifying the results of the analysis and any detected RSIs or any identified IMDs. In some embodiments of the invention, the warning sign may also include a box, circle, or other highlighting of the area of the scan where the suspected RSI or IMD is located. In step 312, the image with any warning indicators is inserted into the radiologist work-list. In some embodiments, the work-list software pushes the most immediate RaPID Response X-ray to the top of the list so that real time operations and emergency room operations are the first X-rays analyzed by the attending radiologist. In other embodiments, the urgency of the X-ray evaluation may be communicated through e-mail, SMS or text messaging, paging systems and the like. In step 314, if the physician/radiologist analysis indicates the presence of an RSI or identification of an IMD then the congruence of the CADe and physician/radiologist evaluation is communicated back to the originating location.

Once the IMD or RFO's is suspected and XR is needed the protocol of embodiments of the present invention includes following specific steps and/or combination of elements:

Once the RaPID Response X-ray is ordered the x-ray technician has available an XR plate with Wi-Fi capabilities in order for images to be instantaneously uploaded to PACS. This eliminates any time delay in the process of manually feeding a plate into a reader which is frequently not immediately available next to the OR suite or emergency department. This uploading process also identifies the new image to the PACS as being of the RaPID Response type, so that appropriate enhancements to the processing of the image are attend to.

Once the portable XR machine is in the operating room and wireless XR plate is in the appropriate position/beneath the patient/the specific settings of the portable XR machine (including kV and mAs) are applied rather then using a standard setting for chest or abdomen XR. These IMD/RSI specific settings increase the chance of having a good quality contrast image of the IMD or RSI. Therefore, any deviation from the standard XR settings such as CXR or KUB predetermined settings with the intention to provide better contrast for identification of RSI or IMD is part of this process. In one embodiment, the exemplary ranges of XR settings based on patient and physical characteristics of the RSIs and IMDs are based on the use of specific XR and processing equipment and may vary between different types of units, and are presented in Table 1:

TABLE 1
X-ray settings for optimal visualization of RSIs and/or IMDs.

| | | KUB | Cx-ray |
|---|---|---|---|
| Normal | NORMAL | kV: 70-80 | kV: 75-85 |
| circumstances | | mAs: 40-80 | mAs: 3.6-8 |
| | OBESE | kV: 80-90 | kV: 90-95 |
| | | mAs: 100-160 | mAs: 5-12 |
| RFO's/sponge | NORMAL | kV: 70-80 | kV: 75-85 |
| or needle case | | mAs: 40-80 | mAs: 3.6-8 |
| | OBESE | kV: 80-90 | kV: 90-95 |
| | | mAs: 100-160 | mAs: 5-12 |

In addition, before the image is taken a specific textual image denominator is assigned to the image—alerting physicians who analyze the image about any RSIs or IMDs and providing information regarding the type of miscount/needle, sponge, or instrument, OR phone call back number, and surgeon's pager. This eliminates the need for the phone call from the OR or ED to the radiologist specifying the type of object for which the image is being searched.

The illustrative embodiment referenced in Table 1 involved a Siemens Axiom Luminos TF with mobile Flat Detector digital radiography system. To evaluate the optimal settings for XR images to show RSI's, combinations of settings were used for various RSI imbedded human-analog (phantom) set-ups. Phantom RSI's were placed on or underneath the sectional imaging phantom (thorax) and then three consecutive images of the same set-up were taken with different parameter settings (High, Medium, Low technique). In this illustrative embodiment, three factors that were varied: the sponge type and x-ray machine parameters of Voltage and Current-exposure. The parameter setting range for voltage (measured in kilovoltage—kV) was 50-125 kV and for current (measured in milliampere-second—mAs) was 1-400 mAs. The illustrative optimal parameter settings were determined in conjunction with a highly trained technician.

Once the image has been taken, in embodiments of the invention a specific optimization of the PACS workflow is specified that automatically puts any RaPID Response X-ray images on the top of the radiologist's workflow list images file with RSIs/IMDs denominator and visible alert.

Usage of computer assisted detection (CADe) software solutions for RSIs detection and/or IMD identification improves the accuracy of detection and decrease time needed for image analysis by radiologist. Computer vision is superior to the human eye in the detection of defined pre-set objects. Embodiments of the invention include software that is based on a novel system using complex pattern recognition algorithms—combining elements of artificial intelligence with digital image processing—to detect RSIs or identify IMDs on medical images. This system analyzes all the images and if any RSIs are detected or IMDs are identified, the system then inserts alert sign over the area of the image with suspected RSIs and/or IMDs.

If a radiologist's analysis is congruent with the CADe software findings—positively detecting RSI or identifying IMD an effective critical information alert is conveyed—the radiologist calls the number assigned to the image while software will also trigger pager alert sent to OR control desk, OR circulating nurse, and attending surgeon. In addition, embodiments of the software automatically provide a link in-patient EMR to the specific PACS images so that OR circulating nurse may pull specific set of images on the computer screen in the OR quickly. In this way, a surgeon may be provided better orientation clues as to where in the operating field any RFO's are located.

The combination of these steps provides a more efficient approach in identification of the IMD or detection of the RFO's. Three possible scenarios are typical:

One scenario involves the RaPID software platform not being integrated into the PACS system but available on portable x-ray machine or as a separate software application—so the physician has the option to activate a RaPID software procedure for analysis of an OR XR image on the screen of the portable XR machine. Once the technician arrives to take XR plate that stores the DICOM image, the technician may ask: “Would you like a RAPID Response X-ray?” This gives the physician that option to choose. When the image is taken through the RaPID Response X-ray process, resulting in the running of a CADe application against the OR XR image, that image is returned to either the operating room or emergency department physician or radiologist, with the image having appropriate markings (e.g., either that nothing was detected or that there were one or more RSIs and/or IMDs identified). This can be show either on the screen of the portable XR machine or bu using hospital computers/electronic medical record interface.

Another scenario involves the RaPID patient safety and quality assurance platform/CADe software with CADe software being integrated with the PACS. Images taken under the RaPID Response x-ray process will have automatically be subject to the CADe application and the resulting image appropriately notated. The above-described process will be routinely used when searching for the RFO's or trying to identify IMD's.

A third scenario involves the Emergency Room (ER), where either of the preceding scenarios applies in the ER context. For example: a patient arrives in the ER and the physicians are in need of identifying any of the patient's IMD's. If the RaPID patient safety and quality assurance platform/CADe software is not integrated within the PACS the first mentioned scenario 1 may apply and if integrated with the PACS the second mentioned scenario may apply.

In several embodiments of the invention, a hospital PACS is relied upon. Furthermore, such embodiments would optionally have the latest generation of imaging devices such as a portable flat-plate XR machine or a C-arm XR machine with processing unit. The process of workflow integration requires the interaction with hospital protocols such as interfacing with hospital count policies and procedures as well as procedures relating to RSIs or IMDs discovery. In addition, embodiments of the invention also interface with the radiological department protocols on managing workflow for image retrieval for examination and evaluation of XR using RaPID Medical Technologies' solutions.

Thus, embodiments of the invention impact several aspects of conventional medical protocols, particularly in the OR and EM contexts. One aspect involves providing an additional step for the main procedure, optionally prior to any procedure to identify any IMD's, or optionally before preparing the patient for ending the procedure, the additional step involving an XR or other scan of the patient is made for the specific purpose of identifying IMDs and/or detecting RSIs. This aspect involves both a protocol and mechanical alteration. The protocol alteration involves having the XR or scan taken by the medical staff in a way to accentuate the entire area of the medical procedure which in some cases may be quite different from the perspective of an XR or scan taken for diagnostic purposes. The mechanical alteration involves the particular settings used by the XR or scan machinery, as particular power levels and spectra may be better at identifying IMDs versus detecting RSis versus assisting diagnosis.

Another aspect involves how such an XR or scan image is processed in the PACS or other image processing workflow. Specifically, RaPID Response X-ray images are given priority in terms of both the computing processing and with the workflow of the radiological image processing system so that both the CADe and radiologist review are performed as quickly as possible given other system constraints. Finally, the medical protocol itself is modified so that when a RaPID Response x-ray is taken, the appropriate computer and radiological review has been taken and the attending physician or surgeon has acknowledged and incorporated the finding of the RaPID Response into the procedure.

Implementation of the Rapid X-ray response process involves having an augmented protocol approved and accepted by hospitals patient safety committee as hospital OR and/or ER policy. The exact implementation of a particular protocol may be specific to a particular health care facility, but should generally include, in some embodiments, a pre-operative scan to confirm initial information about the patient, including without limitation the identification and/or confirmation of the identity and location of any IMDs. In addition such protocols should generally include, in some embodiments, a scan prior to closing up the patient to check or confirm any needle and/or sponge counts, including without limitation, the detection of any RFOs and/or the detection and/or confirmation of the connection of IMDs within the patient. The XR technicians are trained to specifically adjust portable XR machine energy settings to settings that enhance the detection of RFOs and/or IMDs, which in some embodiments are according the recommendations in Table 1 and insert appropriate IMD/RSI denominators, that is to say that in some embodiments certain information about the patient, the procedure, the originating physician, the operative physician, and originating facility, before the XRs are taken. In addition, the OR nursing staff are trained to understand the process of having the additional post-op x-ray step and appropriately react by immediately pulling images on the OR computer screen from the PACS or provide the separate application under the hospital electronic medical record environment. Finally, radiologists are trained and familiarized with the usage of the CADe part of the Rapid x-ray response, including without limitation, the accelerated priority of evaluating RaPID response requests and the denominator information significance.

The XR or scan taken by the medical staff in a way to accentuate the entire area of the medical procedure is quite different from the perspective of a XR or scan taken for diagnostic purposes. More precisely, specific setting of the XR exposure energy for detection of the RSI or identification of IMD needs to be adjusted by programing specific kV and mAs settings before the images are taken. Table 1 summarizes optimal settings based on our current research with some embodiments of the invention. These settings are based on current typical x-ray equipment, and optimal settings for particular type of equipment, types of surgeries, and types of potential RFO's and IMD's may vary over the range of possible combinations. These setting are different from standard settings for CXR or KUB that provide optimal visualization of tissue structures but suboptimal visualization of the RSIs or IMDs. Setting for IMD identification and/or RSI detection are identical as both are optimized to provide highest degree of contrast for non tissue—radiopaque or metal components—in the images, rather than for detail of the diagnostic area of the body. In other embodiments where the scan may be other than an x-ray, for example without limitation an ultrasonic scan or a magnetic resonance imaging scan, then the scanning equipment is adjusted accordingly.

The associated CADe software may be integrated with PACSs either through APIs or as a module resulting in near instantaneous detection of RFOs or identification of IMDs in DIOCOM images as they reach the hosting server. CADe software analyzes OR XR (or any other modality) images and if any RSI is detected or IMD is identified—it inserts an alert sign over the area of the image with suspected RSI/or identified IMD. This alert sign is already embedded in the image as it gets pulled up for analysis under the PACS environment by the surgeon, radiologist, or any other physician. In the case of IMD identification, if the alert sign gets additionally clicked it provides extensive information on IMD's specifications. In some embodiments, CADe software is integrated in the software environment of the portable XR machine in which case alert sign shows up immediately after the image is taken and shown on the portable XR machine screen. Finally, CADe software may exist as a separate application hosted on separate server and as part of hospital electronic medical record environment/applications. In this case any image taken in the ORs are routed to this application that immediately returns pulled images on the computer OR screens with alert sign embedded in the image id IMD identified or RSI detected.

The RaPID CADe software serves as an advisory tool in detection of RSIs and as diagnostic tool in identifying IMDs. It does not disrupt the workflow of PACSs as the analysis of each image occurs before or in conjunction with the acquisition of the image by the PACS, so that the CADe software works seamlessly in coordination with these systems so that there is minimal image processing delay. In one embodiment, for example without limitation where the RaPID CADe software acquires the image from the x-ray scanner and fees the analyzed image to PACS, once the images are pulled up under the PACS environment the RaPID CADe software already has alert signs inserted/embedded into the image. In these embodiments, having either the RaPID CADe software as a separate application or integrated into the portable x-ray machine interface, the RaPID CADe software does acts as an image feeding mechanism and does not interfere with PACS at all. In other embodiments, where the RaPID CADe software is integrated into PACS, the RaPID CADe software is part of the image acquisition and preparation phase of the PACS image storage process, and also relays the results of the RaPID CADe software back to the originating location, i.e. the operating room or emergency room where the scan was taken.

In several embodiments of the invention, RaPID Response X-ray process images are automatically routed to the top of the radiologist workflow list based on image RSI and/or IMD denominators, source—portable XR machine, and OR location. While this is not a mandatory modification of a radiologist's workflow list, achieving one or several of the objects of the present invention is best accomplished in embodiments where such modification is made. The radiologist workflow modification in some embodiments involves modification of the workflow PACS module so that RaPID response X-rays are appropriately prioritized. In other embodiments, such workflow may be directed by a separate radiologist situated workflow software which is modified to accommodate the prioritization of the RaPID Response X-ray image. In addition, the RaPID Response X-ray images, optionally but recommended to be on the top of the workflow list, are additionally flagged clearly showing the urgent status. Upon the images reaching the PACS routing server, the RaPID CADe software analyzes the images and embeds appropriate alert/warning signs if an RSI is detected or an IMD is identified. Therefore once the radiologist pulls up the images under the PACS environment the alert signs are displayed showing if the RaPID CADe software detects an RSI or identifies an IMD.

When a RaPID Response X-ray is taken—images are sent to the PACS server and there analyzed by the CADe software to detect RSI or identify IMD. Once physician pulls up the images under the PACS environment the images already have alert signs if CADe detects RSI or identify IMD. If the physician agrees with the findings of RSI the physician will has an option to trigger an information alert that is conveyed back to the OR and OR front desk. This response is automatically generated with code linking PACs environment/electronic medical record with hospital paging system. If the physician approves IMD identification—this finding will be automatically incorporated into patient's electronic medical record. If the CADe software is incorporated into the portable XR machines or being used as a separate application, such interaction with PACS is implemented as a additional process and may not occur as quickly as with an integrated CADe and PACS.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for performing a medical procedure wherein a scan is taken of the patient specifically for the identification of an implanted medical device prior to the medical procedure.

2. The method of claim 1 wherein the scan image is transmitted to a remote radiologist for evaluation.

3. The method of claim 2 wherein the remote radiologist is also transmitted contact information for an attending physician.

4. The method of claim 2 wherein the transmission of the scan image includes an indication of immediacy to the remote radiologist.

5. The method of claim 1 wherein the scan is taken with a scan plate having telecommunications equipment, and the scan plate sends the scan image via the telecommunications equipment.

6. The method of claim 1 wherein the scan image is also sent to a server at the location of the medical procedure to provide the scan image to an attending physician.

7. A method for performing a medical surgical procedure wherein a scan is taken prior to preparing the patient for concluding the surgical procedure for the specific purpose of detection of a retained foreign object.

8. The method of claim 7 wherein the scan image is transmitted to a remote radiologist for evaluation.

9. The method of claim 8 wherein the remote radiologist is also transmitted contact information for an attending physician.

10. The method of claim 8 wherein the transmission of the scan image includes an indication of immediacy to the remote radiologist.

11. The method of claim 7 wherein the scan is taken with a scan plate having telecommunications equipment, and the scan plate sends the scan image via the telecommunications equipment.

12. The method of claim 7 wherein the scan image is also sent to a server at the location of the medical procedure to provide the scan image to an attending physician.

13. A system for preforming a scan of a surgical area of a patient for the purpose of detection of a retained foreign object, said system comprising:
a computer with a processor, memory, and telecommunications equipment, the computer further having a software program for determining areas of potential retained foreign objects in a scan image;
a scan plate with telecommunications equipment adapted to obtain a scan from proximate a surgical area of a patient and transmitting a scan image to the computer.

14. The system of claim 13 wherein the software program also transmits the scan image to a remote radiologist for evaluation.

15. The system of claim 14 wherein the software program also transmits contact information for an attending physician to the remote radiologist.

16. The system of claim 14 wherein the software program also transmits an indication of immediacy to the remote radiologist.

17. The system of claim 13 further comprising an image server in communication with the computer, the scan image also being sent to the image server of the medical procedure facility to provide the scan image to an attending physician.
